(19) United States
(12) Patent Application Publication
(10) Pub. No.: US 2019/0038130 A1
(43) Pub. Date: Feb. 7, 2019

(54) EYE MOVEMENT DETECTION SYSTEM

(71) Applicant: Intel Corporation, Santa Clara, CA (US)

(72) Inventor: Sean Jude William Lawrence, Bangalore (IN)

(73) Assignee: Intel Corporation, Santa Clara, CA (US)

(21) Appl. No.: 16/147,196

(22) Filed: Sep. 28, 2018

(51) Int. Cl. A61B 3/113; G06F 3/01

(52) U.S. Cl. CPC A61B 3/113; A61B 5/6821; G06F 3/013

(57) ABSTRACT

Particular embodiments described herein provide for an eye movement detection system that can be configured to determine that a pressure in an ear of a user has changed, determine that the pressure change indicates that the eyes of the user are going to move, predict a direction of change of the eye of the user, and track at least one eye of the user based, at least partially, on the determined pressure change.

### TECHNICAL FIELD

This disclosure relates in general to the field of eye tracking devices, and more particularly, to an eye movement detection system.

### BACKGROUND

End users have more electronic device choices than ever before. A number of prominent technological trends are currently afoot, and these trends are changing the electronic device landscape. Some of the technological trends involve eye tracking. Eye tracking is the process of measuring either the point of gaze (where a user is looking) or the motion of an eye relative to the head or the user. An eye tracker is a device for measuring eye positions and eye movement and eye trackers are used in research on the visual system, in psychology, in psycholinguistics, marketing, as an input device for human-computer interaction, in product design, etc.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 is a simplified block diagram of a system to help facilitate eye movement detection system in accordance with an embodiment of the present disclosure;

FIG. 2A is a simplified block diagram of a portion of a system to help facilitate an eye movement detection system in accordance with an embodiment of the present disclosure;

FIG. 2B is a simplified block diagram of a portion of a system to help facilitate an eye movement detection system in accordance with an embodiment of the present disclosure;

FIG. 3 is a simplified block diagram of a portion of a system to help facilitate an eye movement detection system in accordance with an embodiment of the present disclosure;

FIG. 4 is a simplified block diagram of a portion of a system to help facilitate an eye movement detection system in accordance with an embodiment of the present disclosure;

FIG. 5 is a simplified block diagram of a portion of a system to help facilitate an eye movement detection system in accordance with an embodiment of the present disclosure;

FIG. 6 is a simplified block diagram of a portion of a system to help facilitate an eye movement detection system in accordance with an embodiment of the present disclosure;

FIG. 7 is a simplified block diagram of a portion of a system to help facilitate an eye movement detection system in accordance with an embodiment of the present disclosure;

FIG. 8 is a simplified block diagram of a portion of a system to help facilitate an eye movement detection system in accordance with an embodiment of the present disclosure;

FIG. 9 is a simplified flowchart illustrating potential operations that may be associated with the communication system in accordance with an embodiment;

FIG. 10 is a simplified flowchart illustrating potential operations that may be associated with the communication system in accordance with an embodiment;

FIG. 11 is a simplified flowchart illustrating potential operations that may be associated with the communication system in accordance with an embodiment;

FIG. 12 is a block diagram illustrating an example computing system that is arranged in a point-to-point configuration in accordance with an embodiment;

FIG. 13 is a simplified block diagram associated with an example ARM ecosystem system on chip (SOC) of the present disclosure; and

FIG. 14 is a block diagram illustrating an example processor core in accordance with an embodiment.

The FIGURES of the drawings are not necessarily drawn to scale, as their dimensions can be varied considerably without departing from the scope of the present disclosure.

### DETAILED DESCRIPTION OF EMBODIMENTS

The following detailed description sets forth examples of apparatuses, methods, and systems relating to a system to help facilitate the identification of malware in accordance with an embodiment of the present disclosure. Features such as structure(s), function(s), and/or characteristic(s), for example, are described with reference to one embodiment as a matter of convenience; various embodiments may be implemented with any suitable one or more of the described features.

In the following description, various aspects of the illustrative implementations will be described using terms commonly employed by those skilled in the art to convey the substance of their work to others skilled in the art. However, it will be apparent to those skilled in the art that the embodiments disclosed herein may be practiced with only some of the described aspects. For purposes of explanation, specific numbers, materials, and configurations are set forth in order to provide a thorough understanding of the illustrative implementations. However, it will be apparent to one skilled in the art that the embodiments disclosed herein may be practiced without the specific details. In other instances, well-known features are omitted or simplified in order not to obscure the illustrative implementations.

In the following detailed description, reference is made to the accompanying drawings that form a part hereof wherein like numerals designate like parts throughout, and in which is shown, by way of illustration, embodiments that may be practiced. It is to be understood that other embodiments may be utilized and structural or logical changes may be made without departing from the scope of the present disclosure. Therefore, the following detailed description is not to be taken in a limiting sense. For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

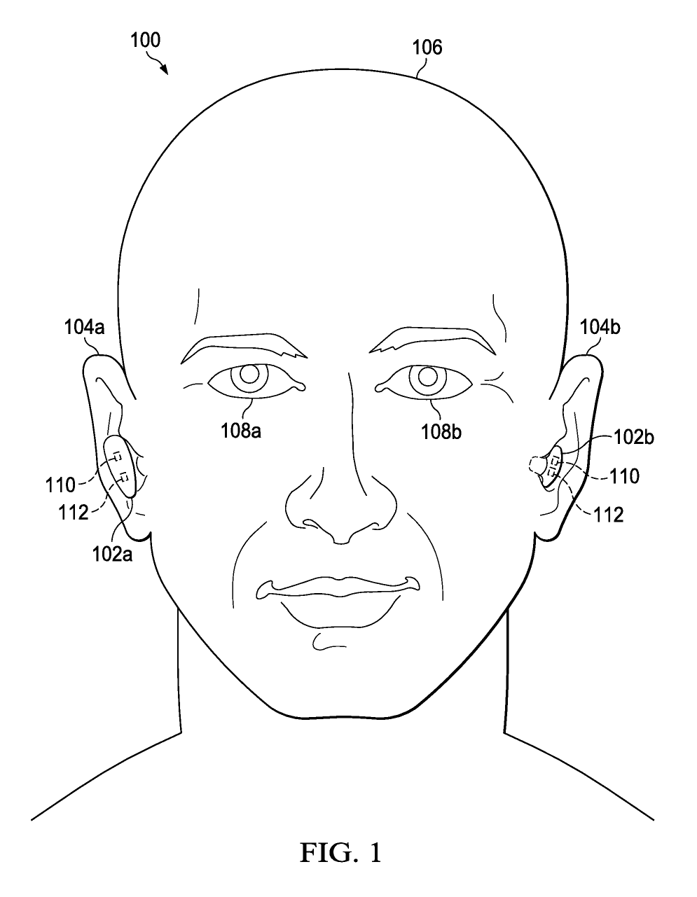

FIG. 1 is a simplified block diagram of an eye movement detection system 102 in accordance with an embodiment of the present disclosure. As illustrated in FIG. 1, a user 100 can include ears 104a and 104b, a head 106, and eyes 108a and 108b. In an example, ear 104a is a right ear on head 106 of user 100 and eye 108a is a right eye on head 106 of user 100. Also, ear 104b is a left ear on head 106 of user 100 and eye 108b is a left eye on head 106 of user 100. An embodiment of eye movement detection system 102 can include eye movement detection system 102a and 102b. Each eye movement detection system 102a and 102b can include an ear pressure based eye movement detection engine 110 and an eye tracking engine 112.

When eyes 108a and 108b move, a pressure change occurs in ears 104a and 104b. The pressure changes indicate when user 100 looks left, right, up down, etc. The pressure change in ears 104a and 104b can begin as early as ten milliseconds before eyes 108a and 108b started to move and the pressure change can continue for a few tens of milliseconds after eyes 108a and 108b have stopped moving. Ear pressure based eye movement detection engine 110 can be configured to detect the change in pressure and communicate a signal to eye tracking engine 112 to help eye tracking engine 112 track eyes 108a and 108b of user 100. In an example, ear pressure based eye movement detection engine 110 can be configured to detect the change in pressure and based on the detected change in ear pressure, eye movement detection system 102a and/or 102b can power a camera or bring a camera from a low power state to an active power state, activate a camera and a predictive eye tracking system, start recording video frames, start recording video frames and activate predictive eye tracking, etc.

More specifically, a few milliseconds before eye 108a moves, a pressure change occurs in ear 104a. Ear pressure based eye movement detection engine 110 in eye movement detection system 102a can be configured to detect the change in pressure in ear 104a and communicate a signal to eye tracking engine 112 in eye movement detection system 102a to help eye tracking engine 112 track eye 108a. Also, a few milliseconds before eye 108b moves, a pressure change occurs in ear 104b. Ear pressure based eye movement detection engine 110 in eye movement detection system 102b can be configured to detect the change in pressure in ear 104b and communicate a signal to eye tracking engine 112 in eye movement detection system 102b to help eye tracking engine 112 track eye 108b. When user 100 turns head 106 but does not move eyes 108a and 108b, the pressure change does not occur.

For purposes of illustrating certain example techniques of the eye movement detection system, the following foundational information may be viewed as a basis from which the present disclosure may be properly explained. Eye tracking refers to the process of measuring where a user is looking, also known as our point of focus. Eye tracking systems work by analyzing the movement of a user's eyes and responds by performing some function. These measurements are carried out by an eye tracker that records the position of the eyes and the movements they make. Eye tracking systems record the location of the eyes or point of focus and eye movements in relation to the environment and are typically based on the optical tracking of corneal reflections, known as pupil center corneal reflection (PCCR). These systems generally include a camera that is always on and tracking the movement of the user's eyes. Since determining the position of the eyes is done after the eyes have moved, there is always an inherent latency associated with such systems. As a result, in order to minimize the latency, the cameras need to run at a high frame per second (FPS) rate (e.g., a rate of 120 FPS) in order to reduce the inherent latency. Unfortunately, the high FPS rate can have a negative impact to power requirements for the system. Contrary to this, eye movement is typically much lower than normal capture rates so some systems run at low capture rates to conserve power and processing cycles. Generally, the speed of eye motion is not as high as a video frame rate of thirty (30) FPS, which is the frame rate used in general live video. The eye cannot move as fast as thirty (30) motions per second and not all captured live video frames need to be processed for eye detection because the same eye movement will be captured on multiple subsequent video frames. However, a low capture rate increases the response latency of the system to eye position change proportionately. What is needed is a system and method to help an eye tracking system anticipate when an eye of a user is going to move.

A system and method to help enable an eye movement detection system, as outlined in FIG. 1 can resolve these issues (and others). Eye movement detection system 102 can be configured to detect pressure changes in a user's ears and use the pressure changes to anticipate when the user's eyes are going to move. In an example, when the eyes of the user are not moving, a camera and other eye tracking elements can be put into a low power state. Eye movement detection system 102 can anticipate when the user's eyes are going to move and bring the camera and other eye tracking elements out of the low power state to track movement of the user's eyes. In addition, based on the detected change in pressure of the user's ears, eye movement detection system 102 can power a camera or bring a camera from a low power state to an active power state, activate a camera and a predictive eye tracking system, start recording video frames, start recording video frames and activate predictive eye tracking, etc.

In another example, the detected pressure change can be used by an eye tracking system that does not include a camera and the detected pressure changes can be used to determine a direction of eye movement and to track the user's eyes. For example, eye movement detection systems 102a and 102b can be in communication with each other and can help to determine a proximate location and/or direction of focus of eyes 108a and 108b based on pressure changes in the user's ears. As used herein, the phrase “determine a direction of eye movement,” “eye tracking,” “focus of the eyes,” “track the user's eyes,” and similar phrases mean to track where the user is looking.

The ear pressure in the user's ears is directly linked to eye movement and will change before eyes 108a and 108b move. Eye movement detection system 102a and 102b can be configured to analyze ear pressure changes to predict and anticipate eye movement. In an example, each of eye movement detection systems 102a and 102b can include one or more microphones and the one or more microphones can be inserted into and/or around a user's ears to detect sound caused by the pressure change that occurs before, during, and after eye movement.

When the user looks left, the ear drum of the user's left ear gets pulled further into the ear and the ear drum of the user's right ear is pushed out and then both swing back and forth a few times, causing pressure changes in the ear and a sound to be generated. These pressure changes to the eardrums and the generated sound began as early as ten milliseconds before the eyes even start to move and continued for a few tens of milliseconds after the eyes stopped. In addition, the peripheral hearing system contains several motor mechanisms that allow the brain to modify the auditory transduction process. Movements or tensioning of either the middle ear muscles or the outer hair cells modifies eardrum motion, producing sounds (e.g., as otoacoustic emissions) that can be detected by a microphone placed in and/or around the ear canal. Ear pressure based eye movement detection engine 110 can detect the oscillations synchronized with and covarying with the direction and amplitude of eye movement, especially saccades type eye movement.

There are four basic types of eye movement, saccades, smooth pursuit movements, vergence movements, and vestibulo-ocular movements. Saccades is the movement that occurs when a user shifts visual focus from one place to another and are rapid, ballistic movements of the eyes that abruptly change the point of fixation. They range in amplitude from the small movements made while reading, for example, to the much larger movements made while gazing around a room. Saccades can be elicited voluntarily, but occur reflexively whenever the eyes are open, even when fixated on a target. Smooth pursuit movements are much slower tracking movements of the eyes. Such movements are under voluntary control in the sense that the observer can choose whether or not to track a moving stimulus but most people who try to move their eyes in a smooth fashion without a moving target simply make saccade eye movements. Vergence movements align each eye with targets located at different distances from the user. Unlike other types of eye movements in which the two eyes move in the same direction, vergence eye movements involve either a convergence or divergence of the lines of sight of each eye to see an object that is nearer or farther away. Vestibulo-ocular movements stabilize the eyes relative to the external world, thus compensating for head movements. These reflex responses prevent visual images from “slipping” on the surface of the retina as head position varies.

As discussed in the article title “The Eardrums Move When the Eyes Move: A Multisensory Effect on the Mechanics of Hearing.” (Gruters, Kurtis G., et al. “The Eardrums Move When the Eyes Move: A Multisensory Effect on the Mechanics of Hearing.” PNAS, National Academy of Sciences, 23 Jan. 2018, www.pnas.org/content/early/2018/01/22/1717948115) herein incorporated by reference, microphone measurements performed during a saccadic eye movement task to visual targets indicates that the eardrum moves in conjunction with the eye movement. The eardrum motion is oscillatory and begins as early as ten milliseconds before saccade onset. These eardrum movements, referred to as eye movement-related eardrum oscillations (EMREOs), occur in the absence of a sound stimulus. The amplitude and phase of the EMREOs depends on the direction and horizontal amplitude of the saccade. The EMREOs last throughout the saccade and well into subsequent periods of steady fixation.

The mechanisms underlying EMREOs may be related to binaural cues that aid the brain in evaluating the relationship between visual and auditory stimulus locations as the eyes move. Visual information can aid hearing, such as when lip reading cues facilitate speech comprehension. To derive such benefits, the brain must first link visual and auditory signals that arise from common locations in space. In species with mobile eyes (e.g., humans), visual and auditory spatial cues bear no fixed relationship to one another but change dramatically and frequently as the eyes move (e.g., about three times per second over an 80° range of space).

In an example, eye movement detection system 102 can be configured to utilizes the ear pressure changes to optimize the eye tracking. Since ear pressure starts changing about ten milliseconds before the onset of intended eye movement, eye movement detection system 102 can predict when eye movement will occur and in which direction. When eye movement is predicted, eye movement detection system 102 can switch on/begin eye tracking or prime the eye tracking pipeline (e.g., analysis compute cycles that need be assigned for eye tracking) to begin eye tracking only when (or before) eye movement occurs. Search algorithms for the eyes can be focused on the area proximate to the direction the eyes are predicted to be positioned. This allows eye movement detection system 102 to switch off power or put a camera and pipeline in a low power state (e.g., stop capture and retain context in the capture pipeline and sensor) to reduce power when the eyes are not moving. There can also be significant saving in analysis compute cycles as eye movement detection system 102 does not need to track the eyes at times when there is no movement. Additionally, eye movement detection system 102 can be configured to determine when exactly an eye change is going to occur and in which direction. As a result, the camera suddenly becomes a modality that is only needed to tell eye movement detection system 102 where the eye gaze is focused or aligned after eye movement has been detected by the change in ear pressure. If a camera is present in eye movement detection system 102, when the eyes are not moving above a threshold amount, eye movement detection system 102 can be configured to put the camera into a low power state (e.g., an intermediate/standby power state) where power is relatively low. When ear pressure is detected above a threshold amount, recovery time (when eye movement is detected) from the low state to an active capture power state can be quick enough to provide a latency benefit. In addition, during the low power state, the camera can run in an active streaming mode at a very low frame rate (e.g., 0.5 or 1 FPS) and the frame/capture rate can be increased when eye movement is predicted.

In an illustrative example, eye movement detection system 102 can monitor the ears of a user for pressure changes. If the ear pressure change is within a particular threshold, nothing is done and the system continues to measure and analyze ear pressure. The threshold amount is the amount of eye movement above a predetermined pressure and/or a pressure change above a predetermined amount of time (e.g., longer than the pressure change caused by an eye flicker) and can help eliminate the system tracking insignificant and irrelevant saccades eye movement. In addition, the threshold can depend on the application and can be adjusted based on user and/or administrator preference. If significant ear pressure change is detected, the pressure change is determined to correspond to significant eye motion and in an example, a horizontal coordinate of the eye position is predicted. If the head of the user is vertically oriented, an eye search algorithm searches for the eyes in a vertical band/bands around the horizontal coordinate (other orientations would use differently oriented bands). Once detected, normal eye tracking is performed until the eye position remains constant for a defined period of time, after which, eye tracking is stopped and the system continues to detect and predict eye position changes by continuing to analyze ear pressure. The cycle repeats on detection of further ear pressure changes.

Eye movement detection system 102 be may be applied to many form factors that have eye tracking. The ear pressure measurements may be made with associated microphone/sensors integrated on headphone/ear bud form factors that are naturally placed in and/or around the ear of the user. Eye movement detection system 102 may also be applied to head mounted displays (HMDs) which offer a more controlled environment to design ear enclosures within which ear pressure may be measured and analyzed.

Additionally, use of eye movement detection system 102 to monitor ear pressure and determine approximate eye positioning may be used in systems that do not have a camera for eye tracking. For example, eye movement detection system 102 may be integrated into a helmet of a user (e.g., a user riding a 2-wheeler) where instead of integrating a camera in a helmet that would make the helmet very costly, the user's eye positions can be detected using ear pressure and audio or haptic feedback can be provided during times of distraction. In an illustrative example, eye movement detection system 102 can be used to monitor a user's eyes to make sure the eyes are not taken off a target (e.g., a road) and an alert or alarm can sound when the eyes are taken off the target for a period of time (which can indicate a distracted or sleepy driver if the target was the road). In another illustrative example, eye movement detection system 102 can be used by a user in a retail environment. Eye positions (determined by eye movement detection system 102) may be analyzed in combination with other retail environment infrastructure cameras to analyze the actions and movements of the user to provide a better retail experience to the user.

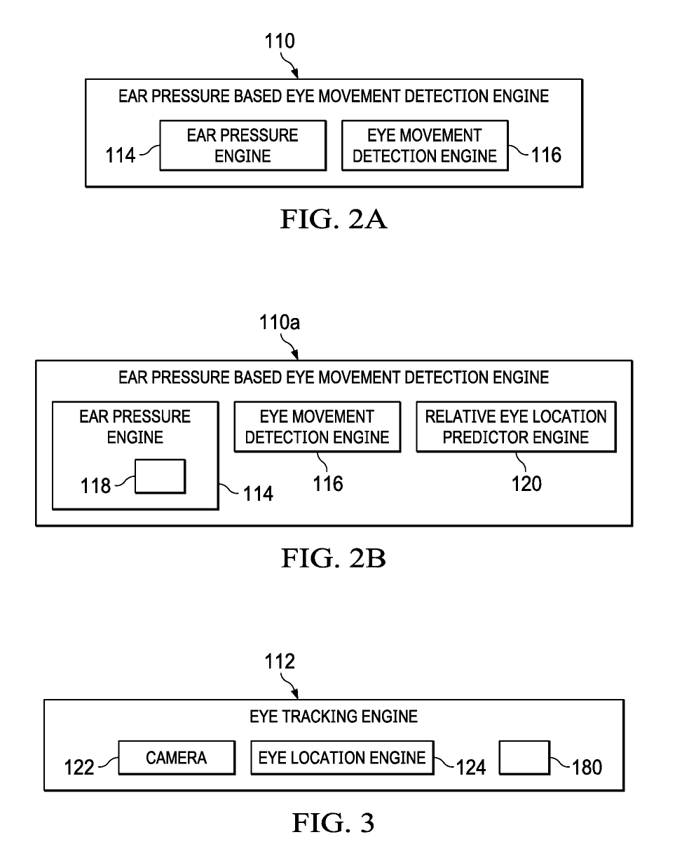

Turning to FIG. 2A, FIG. 2A is a simplified block diagram of a portion of eye movement detection system 102 in accordance with an embodiment of the present disclosure. In an example, ear pressure based eye movement detection engine 110 can include an ear pressure engine 114 and an eye movement detection engine 116. Ear pressure engine 114 can be configured to detect pressure changes in an ear (e.g., ear 104a) of a user. Eye movement detection engine 116 can be configured to determine if the pressure changes in the ear relate to eye movement. For example, ear pressure engine 114 may detect pressure changes in the ear and eye movement detection engine 116 can be configured to determine if the pressure changes are caused by the pressure change during an eye flicker and therefore are below a threshold or if the pressure changes are above a threshold and therefore are related to eye movement where the user is moving their eyes to a different view or area of focus. In addition, eye movement detection engine 116 can be configured to help anticipate eye movement by converting the ear pressure detected by ear pressure engine 114 into a direction the eye will move. In another example, if eye movement detection engine 116 determines that the pressure changes detected by ear pressure engine 114 are above a threshold and therefore are related to eye movement, eye movement detection engine 116 can communicate with eye tracking engine 112 to wake eye tracking engine 112 from a low power state so eye tracking engine 112 can begin tracking the eye of the user. In addition, eye movement detection engine 116 can communicate with eye tracking engine 122 to power a camera or bring a camera from a low power state to an active power state, activate a camera and a predictive eye tracking system, start recording video frames, start recording video frames and activate predictive eye tracking, etc.

Turning to FIG. 2B, FIG. 2B is a simplified block diagram of a portion of eye movement detection system 102 in accordance with an embodiment of the present disclosure. In an example, ear pressure based eye movement detection engine 110a can include ear pressure engine 114, eye movement detection engine 116, and relative eye location predictor engine 120. Ear pressure engine 114 can include a microphone 118.

Microphone 118 can be configured to detect the sound made when the ear pressure changes. Relative eye location predictor engine 120 can be configured to determine a relative eye location of the user. For example, relative eye location predictor engine 120 can be configured to predict the relative location of the eye by converting the ear pressure detected by ear pressure engine 114 into a direction the eye will move relative to where the eye is located. In some implementations, this can be used to track a user's eyes without the need of a camera or other video based eye tracking system. In another example, relative eye location predictor engine 120 can communicate with eye tracking engine 112 to wake eye tracking engine 112 from a low power state and provide data regarding the direction the eye will move and a general area of where the eye may be so eye tracking engine 112 can focus on an area where the eye should be located and begin tracking the eye of the user.

Turning to FIG. 3, FIG. 3 is a simplified block diagram of a portion of eye movement detection system 102 in accordance with an embodiment of the present disclosure. In an example, eye tracking engine 112 can include a camera 122 and an eye location engine 124. In an example, eye tracking engine 112 can be configured to track the movement of an eye (e.g., eye 108a) of a user using camera 122.

Eye tracking is the process of measuring either the point of gaze (where one is looking) or the motion of an eye relative to the head. An eye tracker is a device for measuring eye positions and eye movement. Eye trackers are used in research on the visual system, in psychology, in psycholinguistics, marketing, as an input device for human-computer interaction, and in product design. There are a number of methods for measuring eye movement, and most variants use video images from which the eye position is extracted. Other methods use search coils or are based on an electrooculogram.

In another example, camera 122 may not be present in eye tracking engine 112 and eye location engine 124 can be configured to determine the relative location of the eye. More specifically, relative eye location predictor engine 120 can be configured to predict the relative location of the eye by converting the ear pressure detected by ear pressure engine 114 into a direction the eye will move. Eye location engine 124 can be configured to determine where the eye was located (e.g., eye location engine 124 can store a current location of the eye in memory 180) and using the data from relative eye location predictor engine 120 to track the eye of the user as it moves.

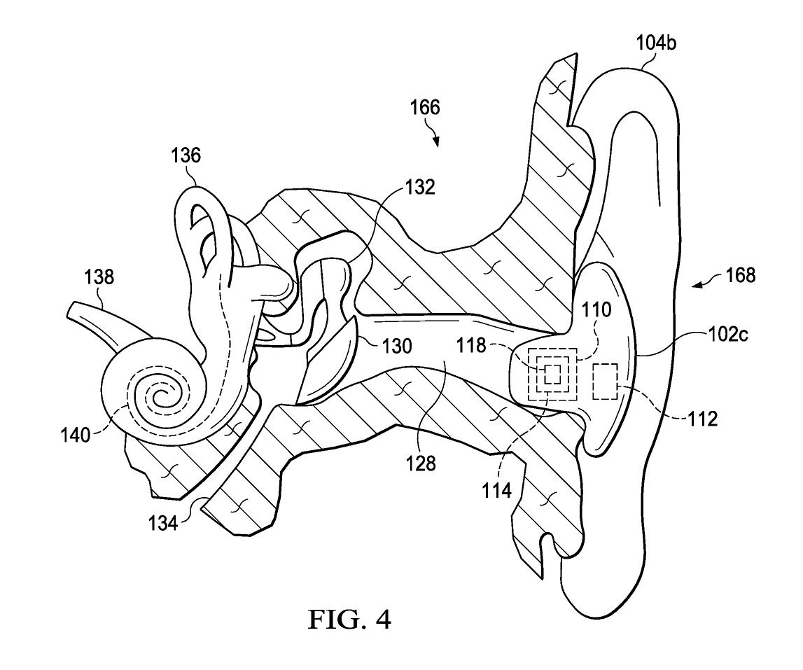

Turning to FIG. 4, FIG. 4 is a simplified block diagram of a portion of an eye movement detection system 102 in accordance with an embodiment of the present disclosure. In an example, ear 104b can have an inner portion 166 and an outer portion 168. Inner portion 166 can include an ear canal 128, an ear drum 130, ossicles 132, eustachian tube 134, cochlea 136, auditory nerve 138, and basilar membrane 140. Basilar membrane 140 is located inside cochlea 136. In an example, eye movement detection system 102a can extend into inner portion 166 of ear 104b (e.g., into ear canal 128).

A user's auditory periphery system possesses at least two means of tailoring its processing in response to descending neural control. First, the middle ear muscles (MEMs), attach to ossicles 132 that connect ear drum 130 to the cochlea 136. Contraction of these muscles tugs on ossicles 132. This modulates middle ear sound transmission and moves ear drum 130. Second, within cochlea 136, the outer hair cells (OHCs) are mechanically active and modify the motion of both basilar membrane 140 and, through mechanical coupling via ossicles 132, ear drum 130. The actions of the MEMs and OHCs affect not only the response to incoming sound but also transmit vibrations backward to the eardrum. Both the MEMs and OHCs are subject to descending control by signals from the central nervous system, allowing the brain to adjust the cochlear encoding of sound in response to previous or ongoing sounds in either ear and based on global factors, such as attention. The collective action of these systems can be measured in real time by eye movement detection system 102c. For example, microphone 118 in ear canal 128 can detect the sound created by pressure changes in inner portion 166 of ear 104b. In another example, ear pressure based eye movement detection engine 110 can be configured to detect sound and/or ear pressure changes within ear 104b using means other than microphone 118 (e.g., pressure sensor, etc.).

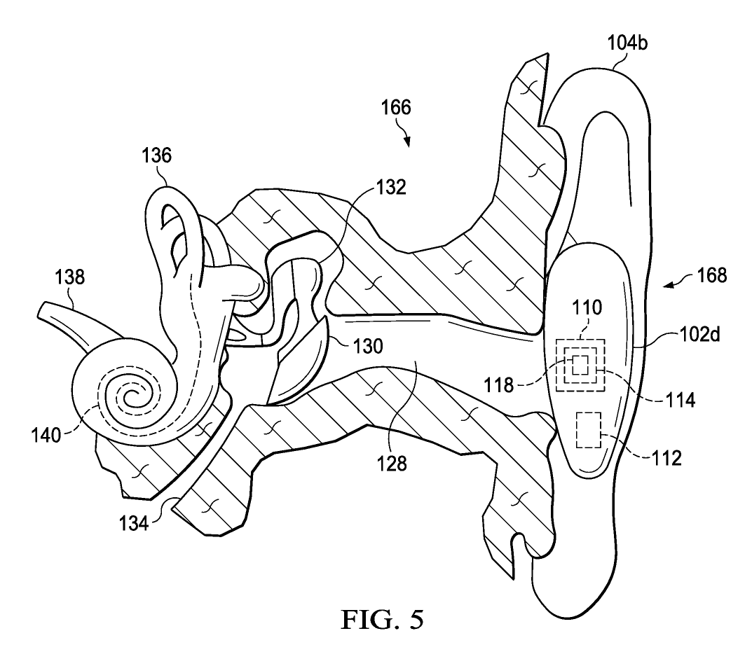

Turning to FIG. 5, FIG. 5 is a simplified block diagram of a portion of an eye movement detection system 102 in accordance with an embodiment of the present disclosure. In an example, ear 104b can include inner portion 166 and outer portion 168. Inner portion 166 can include ear canal 128, ear drum 130, ossicles 132, eustachian tube 134, cochlea 136, auditory nerve 138, and basilar membrane 140. In an example, eye movement detection system 102a does not extend into inner portion 166 of ear 104b (e.g., does not extend into ear canal 128). Microphone 118 outside of ear canal 128 can detect the sound created by pressure changes in inner portion 166 of ear 104b. In another example, ear pressure based eye movement detection engine 110 can be configured to detect sound and/or ear pressure changes within ear 104b using means other than microphone 118 (e.g., pressure sensor, etc.).

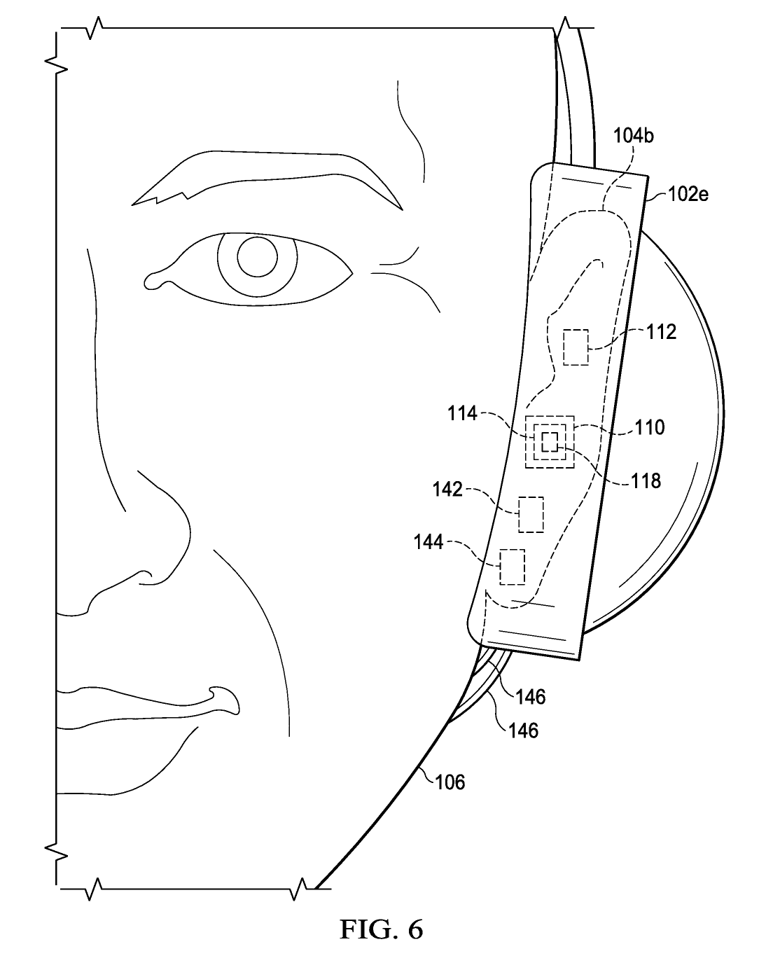

Turning to FIG. 6, FIG. 6 is a simplified block diagram of a portion of an eye movement detection system 102 in accordance with an embodiment of the present disclosure. In an example, eye movement detection system 102e can include ear pressure based eye movement detection engine 110, eye tracking engine 112, wireless communication engine 142, noise cancelling engine 144, and one or more pressure sensors 146. Eye movement detection system 102e can be located over ear 104b. Microphone 118 outside of ear canal 128 can detect the sound created by pressure changes in inner portion 166 of ear 104b. In another example, ear pressure based eye movement detection engine 110 can be configured to detect sound and/or ear pressure changes within ear 104b using means other than microphone 118 (e.g., pressure sensor, etc.).

Wireless communication engine 142 can be configured to wirelessly communicate with an electronic device (e.g., smart phone, wearable, gaming system, virtual reality system, network element, etc.). Noise cancelling engine 144 can be configured to actively reduce noise interference and allow microphone 118 to detect the sound created by pressure changes in inner portion 166 of ear 104b. Active noise reduction, also known as noise cancellation, or active noise reduction (ANR), is a method for reducing unwanted sound by the addition of a second sound specifically designed to cancel the first. For example, noise cancelling engine 144 can be configured to cancel sounds or noise from outside of inner portion 166 of ear 104b (e.g., wind noise or other ambient noise, if the user is listening to music, participating in a virtual reality environment where there is sound, etc.) that may affect microphone 118. In another example, the sounds or noise from outside of inner portion 166 of ear 104b may be used to filter the sound detected by microphone 118 to create a signal that only (or mostly) includes sounds or noise from inside of inner portion 166 of ear 104b. One or more pressure sensors 146 can be located on or near the jaw of the user and can help determine when a pressure change in the ear of the user is due to jaw movement of the user (e.g., the user is swallowing, talking, yawning, etc.) and can be used by ear pressure based eye movement detection engine 110 to determine when the sound and/or ear pressure changes within ear 104b are related to eye movement.

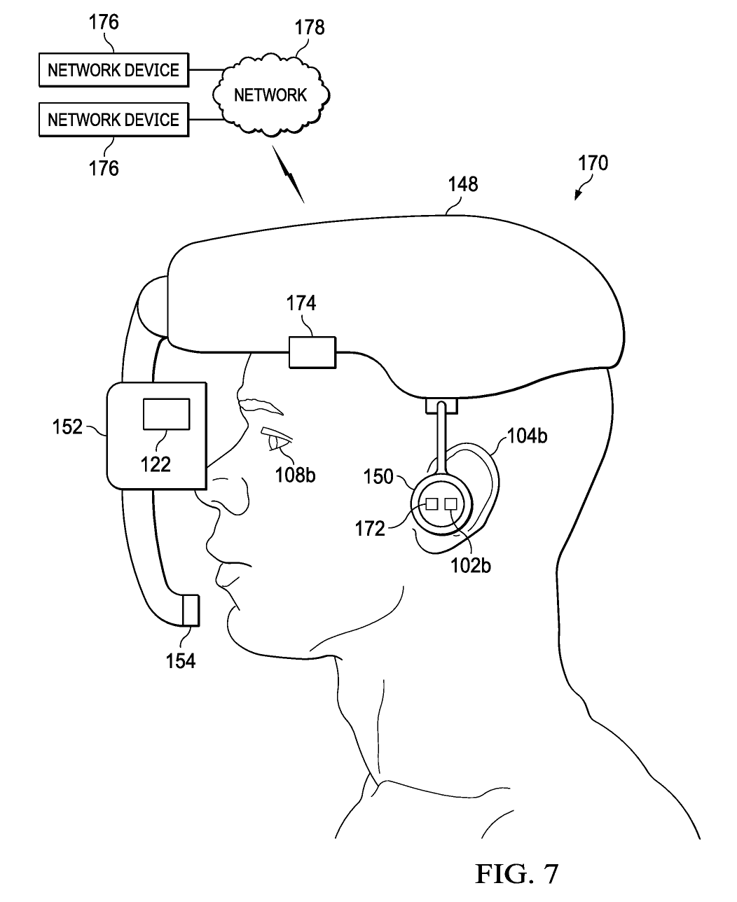

Turning to FIG. 7, FIG. 7 is a simplified block diagram of a portion of an eye movement detection system 102 in accordance with an embodiment of the present disclosure. In an example, eye movement detection system 102b (and 102a not shown) can be included in a wearable 170 such as a virtual reality helmet or headgear. Wearable 170 can include camera 122, a head piece 148, an ear piece 150, a screen or display 152, and a microphone 154. Ear piece 150 can include eye movement detection system 102e and a speaker 172. In an example, wearable 170 can include network engine 174. Network engine 174 can be in communication with one or more network devices 176 using network 178. Network engine 174 may be a wired or wireless communication engine.

Eye movement detection system 102b can be configured to utilizes ear pressure to help optimize the eye tracking. Since ear pressure starts changing about ten milliseconds before the onset of intended eye movement, eye movement detection system 102b can predict when eye movement will occur and in which direction in the horizontal plane the eye will be located and switch on/begin eye tracking or prime the eye tracking pipeline (analysis compute cycles that need be assigned for eye tracking) to begin eye tracking only when (or before) eye movement occurs. Search algorithms for the eyes can be focused on the area proximate to the direction the eyes would be positioned. This allows eye movement detection system 102b to switch off power or put camera 122 in a low power state (stop capture and retain context in the capture pipeline and sensor) to reduce power when the eyes are not moving. Recovery time (when eye movement is detected) from the reduced power state to an active capture state can be quick enough to provide a latency benefit. In addition, camera 122 can run in an active streaming mode at very low frame rate (0.5 or 1 FPS) and frame/capture rate can be increased when eye movement is predicted by eye movement detection system 102b.

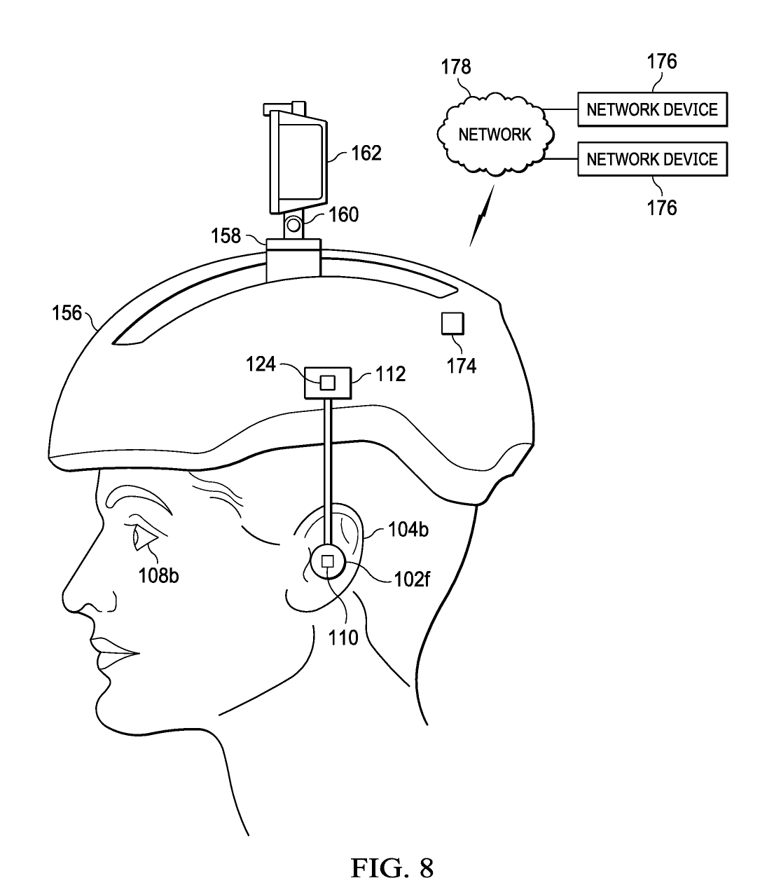

Turning to FIG. 8, FIG. 8 is a simplified block diagram of a portion of an eye movement detection system 102 in accordance with an embodiment of the present disclosure. In an example, eye movement detection system 102f (and 102a not shown) can be included in a helmet 156. Helmet 156 may be headgear or a protective helmet. Helmet 156 can include eye movement detection system 102f, eye tracking engine 112, a device positioning engine 158, and a device mount 160. Eye movement detection system 102f can include ear pressure based eye movement detection engine 110. Eye tracking engine 112 can include eye location engine 124.

In an example, a device 162 (e.g., a video camera as illustrated in FIG. 8) can be coupled to device mount 160. Using ear pressure based eye movement detection engine 110, eye movement detection system 102f can be configured to track the ear pressure changes that occur during movement of the eyes (e.g., eye 108b) of the user. This information can be communicated to eye tracking engine 112 and used by eye location engine 124 to help track the eyes of the user. Device positioning engine 158 can use device mount 160 to adjust device 162 so that device 162 follows the direction of the eyes of the user. In another example, eye movement detection system 102 can be used to monitor a user's eyes to make sure the eyes are not taken off a target (e.g., a road) and an alert or alarm can sound when the eyes are taken off the target for a period of time (which can indicate a distracted or sleepy driver if the target was the road).

In an example, helmet 156 can include network engine 174. Network engine 174 can be in communication with one or more network devices 176 using network 178. Network engine 174 may be a wired or wireless communication engine.

Elements of FIGS. 7 and 8 may be coupled to one another through one or more interfaces employing any suitable connections (wired or wireless), which provide viable pathways for network (e.g., network 178) communications. Additionally, any one or more of these elements of FIGS. 7 and 8 may be combined or removed from the architecture based on particular configuration needs. Network engine 174, network 178, and network devices 176 may include a configuration capable of transmission control protocol/Internet protocol (TCP/IP) communications for the transmission or reception of packets in a network. Network engine 174, network 178, and network devices 176 may also operate in conjunction with a user datagram protocol/IP (UDP/IP) or any other suitable protocol where appropriate and based on particular needs.

Turning to the infrastructure of FIGS. 7 and 8, generally, the system can be implemented in any type or topology of networks. Network 178 represents a series of points or nodes of interconnected communication paths for receiving and transmitting packets of information that propagate through the system. Network 178 offers a communicative interface between nodes, and may be configured as any local area network (LAN), virtual local area network (VLAN), wide area network (WAN), wireless local area network (WLAN), metropolitan area network (MAN), Intranet, Extranet, virtual private network (VPN), and any other appropriate architecture or system that facilitates communications in a network environment, or any suitable combination thereof, including wired and/or wireless communication.

In the system, network traffic, which is inclusive of packets, frames, signals, data, etc., can be sent and received according to any suitable communication messaging protocols. Suitable communication messaging protocols can include a multi-layered scheme such as Open Systems Interconnection (OSI) model, or any derivations or variants thereof (e.g., Transmission Control Protocol/Internet Protocol (TCP/IP), user datagram protocol/IP (UDP/IP)). Additionally, radio signal communications over a cellular network may also be provided in the system. Suitable interfaces and infrastructure may be provided to enable communication with the cellular network.

The term “packet” as used herein, refers to a unit of data that can be routed between a source node and a destination node on a packet switched network. A packet includes a source network address and a destination network address. These network addresses can be Internet Protocol (IP) addresses in a TCP/IP messaging protocol. The term “data” as used herein, refers to any type of binary, numeric, voice, video, textual, or script data, or any type of source or object code, or any other suitable information in any appropriate format that may be communicated from one point to another in electronic devices and/or networks. Additionally, messages, requests, responses, and queries are forms of network traffic, and therefore, may comprise packets, frames, signals, data, etc.

Network devices 176 can each be a desktop computer, laptop computer, mobile device, personal digital assistant, smartphone, tablet, network appliances, servers, cloud services, routers, switches, gateways, bridges, load balancers, or any other suitable device, component, element, or object operable to exchange information in a network environment. A server can be a network element such as a server or virtual server and can be associated with clients, customers, endpoints, or end users wishing to initiate a communication via some network (e.g., network 178). The term ‘server’ is inclusive of devices used to serve the requests of clients and/or perform some computational task on behalf of clients. Cloud services may generally be defined as the use of computing resources that are delivered as a service over a network, such as the Internet. Typically, compute, storage, and network resources are offered in a cloud infrastructure, effectively shifting the workload from a local network to the cloud network. Network devices 176 may include any suitable hardware, software, components, modules, or objects that facilitate the operations thereof, as well as suitable interfaces for receiving, transmitting, and/or otherwise communicating data or information in a network environment. This may be inclusive of appropriate algorithms and communication protocols that allow for the effective exchange of data or information.

In regards to the internal structure, eye movement detection system 102 can include memory elements for storing information to be used in the operations outlined herein. Eye movement detection system 102 may keep information in any suitable memory element (e.g., disk, random access memory (RAM), read-only memory (ROM), erasable programmable ROM (EPROM), electrically erasable programmable ROM (EEPROM), application specific integrated circuit (ASIC), etc.), software, hardware, firmware, or in any other suitable component, device, element, or object where appropriate and based on particular needs. Any of the memory items discussed herein should be construed as being encompassed within the broad term ‘memory element.’ Moreover, the information being used, tracked, sent, or received in eye movement detection system 102 could be provided in any database, register, queue, table, cache, control list, or other storage structure, all of which can be referenced at any suitable timeframe. Any such storage options may also be included within the broad term ‘memory element’ as used herein.

In certain example implementations, the functions outlined herein may be implemented by logic encoded in one or more tangible media (e.g., embedded logic provided in an ASIC, digital signal processor (DSP) instructions, software (potentially inclusive of object code and source code) to be executed by a processor, or other similar machine, etc.), which may be inclusive of non-transitory computer-readable media. In some of these instances, memory elements can store data used for the operations described herein. This includes the memory elements being able to store software, logic, code, or processor instructions that are executed to carry out the activities described herein.

In an example implementation, eye movement detection system 102 may include software modules (e.g., ear pressure based eye movement detection engine 110, eye tracking engine 112, ear pressure engine 114, eye movement detection engine 116, relative eye location predictor engine 120, eye location engine 124, etc.) to achieve, or to foster, operations as outlined herein. These modules may be suitably combined in any appropriate manner, which may be based on particular configuration and/or provisioning needs. In example embodiments, such operations may be carried out by hardware, implemented externally to these elements, or included in some other network device to achieve the intended functionality. Furthermore, the modules can be implemented as software, hardware, firmware, or any suitable combination thereof. These elements may also include software (or reciprocating software) that can coordinate with other network elements in order to achieve the operations, as outlined herein.

Additionally, eye movement detection system 102 may include a processor that can execute software or an algorithm to perform activities as discussed herein. A processor can execute any type of instructions associated with the data to achieve the operations detailed herein. In one example, the processors could transform an element or an article (e.g., data) from one state or thing to another state or thing. In another example, the activities outlined herein may be implemented with fixed logic or programmable logic (e.g., software/computer instructions executed by a processor) and the elements identified herein could be some type of a programmable processor, programmable digital logic (e.g., a field programmable gate array (FPGA), an EPROM, an EEPROM) or an ASIC that includes digital logic, software, code, electronic instructions, or any suitable combination thereof. Any of the potential processing elements, modules, and machines described herein should be construed as being encompassed within the broad term ‘processor.’

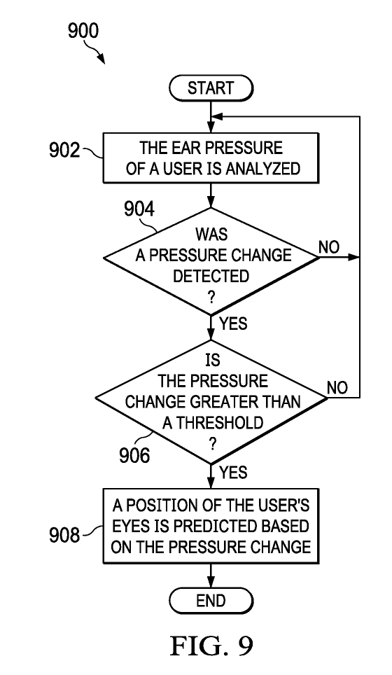

Turning to FIG. 9, FIG. 9 is an example flowchart illustrating possible operations of a flow 900 that may be associated with an eye movement detection system. In an embodiment, one or more operations of flow 900 may be performed by eye movement detection system 102a and 102b, ear pressure based eye movement detection engine 110, eye tracking engine 112, ear pressure engine 114, eye movement detection engine 116, relative eye location predictor engine 120, and eye location engine 124. At 902, the ear pressure of a user is analyzed. At 904, the system determines if a pressure change was detected. If a pressure change was not detected, then the ear pressure of the user continues to be analyzed, as in 902. If a pressure change was detected, then the system determines if the pressure change is greater than a threshold, as in 906. If the pressure change is not greater than a threshold then the ear pressure of the user continues to be analyzed, as in 902. If the pressure change is greater than a threshold, then a position of the user's eyes is predicted based on the pressure change, as in 908. The threshold amount is the amount of eye movement above a predetermined pressure and/or a pressure change for a predetermined amount of time (e.g., longer than the pressure change caused by an eye flicker) and can help eliminate the system tracking insignificant and irrelevant saccades eye movement. In addition, the threshold can depend on the application and can be adjusted based on user and/or administrator preference.

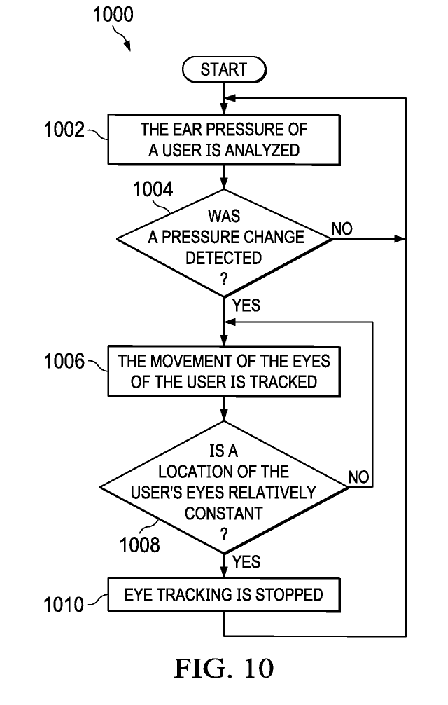

Turning to FIG. 10, FIG. 10 is an example flowchart illustrating possible operations of a flow 1000 that may be associated with an eye movement detection system. In an embodiment, one or more operations of flow 1000 may be performed by eye movement detection system 102a and 102b, ear pressure based eye movement detection engine 110, eye tracking engine 112, ear pressure engine 114, eye movement detection engine 116, relative eye location predictor engine 120, and eye location engine 124. At 1002, the ear pressure of a user is analyzed. At 1004, the system determines if a pressure change was detected. If a pressure change was not detected, then the ear pressure of the user continues to be analyzed, as in 1002. If a pressure change was detected, then the movement of the eyes of the user is tracked, as in 1006. At 1008, the system determines if a location of the user's eyes is relatively constant. If a location of the user's eyes is not relatively constant, then the movement of the eyes of the user continues to be tracked, as in 1006. If a location of the user's eyes is relatively constant, then eye tracking is stopped, as in 1010, and the ear pressure of a user is analyzed, as in 1002. For example, eye tracking engine 112 can be put in a low power state or standby mode where the user's eyes are not tracked. If a camera (e.g., camera 122) is present, the camera may be put in a low power state or standby mode where the user's eyes are not tracked.

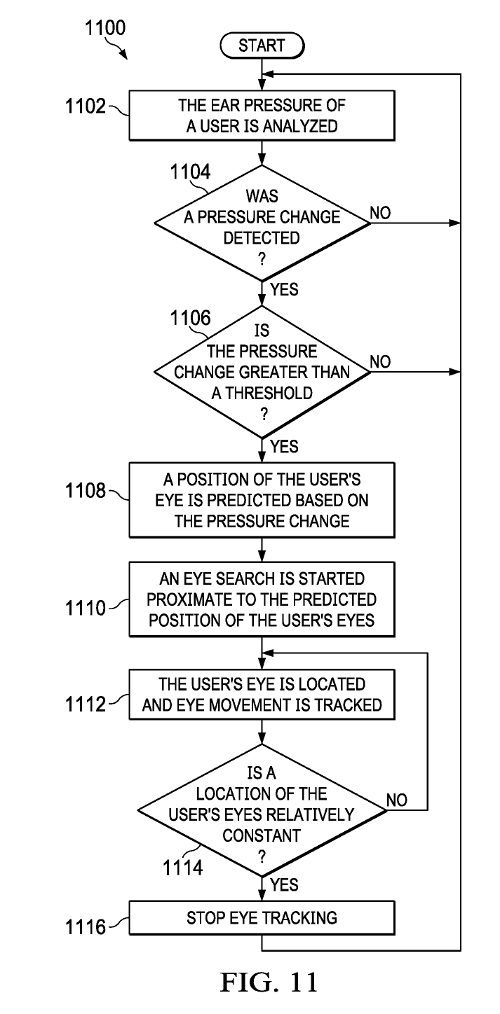

Turning to FIG. 11, FIG. 11 is an example flowchart illustrating possible operations of a flow 1100 that may be associated with an eye movement detection system. In an embodiment, one or more operations of flow 1100 may be performed by eye movement detection system 102a and 102b, ear pressure based eye movement detection engine 110, eye tracking engine 112, ear pressure engine 114, eye movement detection engine 116, relative eye location predictor engine 120, and eye location engine 124. At 1102, the ear pressure of a user is analyzed. At 1104, the system determines if a pressure change was detected. If a pressure change was not detected, then the ear pressure of the user continues to be analyzed, as in 1102. If a pressure change was detected, then the system determines if the pressure change is greater than a threshold, as in 1106. If the pressure change is not greater than a threshold then the ear pressure of the user continues to be analyzed, as in 1102. If the pressure change is greater than a threshold, then a position of the user's eyes is predicted based on the pressure change, as in 1108. At 1110, an eye search is started proximate to the predicted position of the user's eyes. At 1112, the user's eye is located and eye movement is tracked. At 1114, the system determines if a location of the user's eyes is relatively constant. If a location of the user's eyes is not relatively constant, then the movement of the eyes of the user continues to be tracked, as in 1112. If a location of the user's eyes is relatively constant then eye tracking is stopped, as in 1116, and the ear pressure of a user is analyzed, as in 1102.

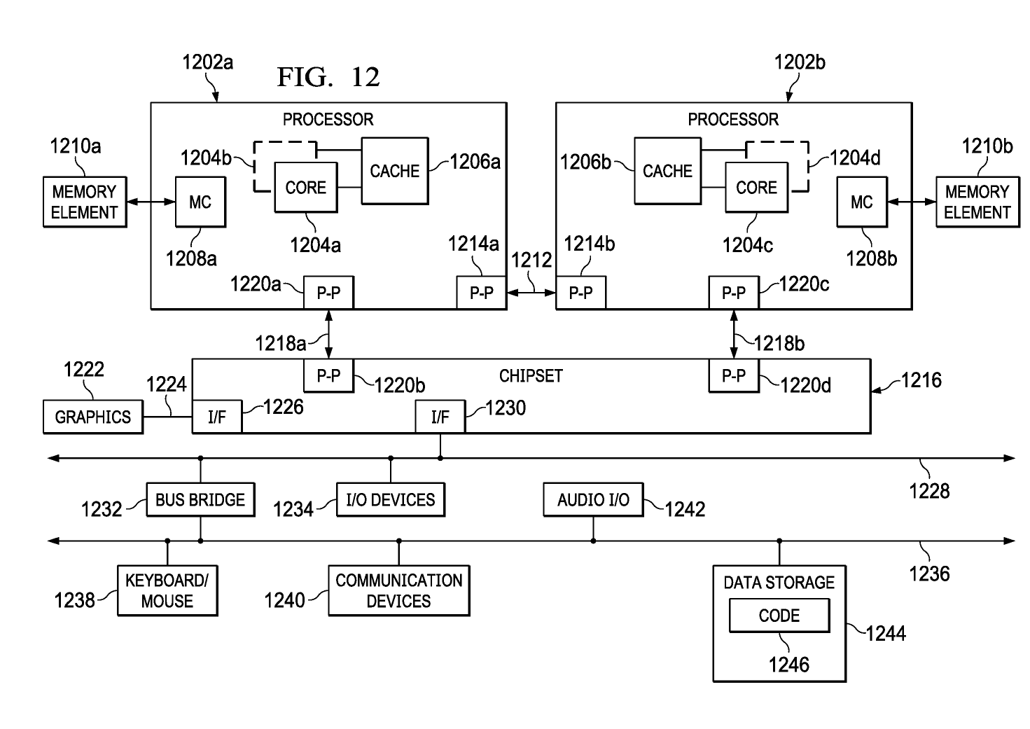

Turning to FIG. 12, FIG. 12 illustrates a computing system 1200 that is arranged in a point-to-point (PtP) configuration according to an embodiment. In particular, FIG. 12 shows a system where processors, memory, and input/output devices are interconnected by a number of point-to-point interfaces. Generally, one or more of the network elements may be configured in the same or similar manner as computing system 1200.

As illustrated in FIG. 12, system 1200 may include several processors, of which only two, processors 1202a and 1202b, are shown for clarity. While two processors 1202a and 1202b are shown, it is to be understood that an embodiment of system 1200 may also include only one such processor. Processors 1202a and 1202b may each include a set of cores (i.e., processors cores 1204a and 1204b and processors cores 1204c and 1204d) to execute multiple threads of a program. The cores may be configured to execute instruction code in a manner similar to that discussed above with reference to FIGS. 1-8. Each processor 1202a and 1202b may include at least one shared cache 1206a and 1206b respectively. Shared caches 1206a and 1206b may each store data (e.g., instructions) that are utilized by one or more components of processors 1202a and 1202b, such as processor cores 1204a and 1204b of processor 1202a and processor cores 1204c and 1204d of processor 1202b.

Processors 1202a and 1202b may also each include integrated memory controller logic (MC) 1208a and 1208b respectively to communicate with memory elements 1210a and 1210b. Memory elements 1210a and/or 1210b may store various data used by processors 1202a and 1202b. In alternative embodiments, memory controller logic 1208a and 1208b may be discrete logic separate from processors 1202a and 1202b.

Processors 1202a and 1202b may be any type of processor and may exchange data via a point-to-point (PtP) interface 1212 using point-to-point interface circuits 1214a and 1214b respectively. Processors 1202a and 1202b may each exchange data with a chipset 1216 via individual point-to-point interfaces 1218a and 1218b using point-to-point interface circuits 1220a-1220d. Chipset 1216 may also exchange data with a high-performance graphics circuit 1222 via a high-performance graphics interface 1224, using an interface circuit 1226, which could be a PtP interface circuit. In alternative embodiments, any or all of the PtP links illustrated in FIG. 12 could be implemented as a multi-drop bus rather than a PtP link.

Chipset 1216 may be in communication with a bus 1228 via an interface circuit 1230. Bus 1228 may have one or more devices that communicate over it, such as a bus bridge 1232 and I/O devices 1234. Via a bus 1236, bus bridge 1232 may be in communication with other devices such as a keyboard/mouse 1238 (or other input devices such as a touch screen, trackball, etc.), communication devices 1240 (such as modems, network interface devices, or other types of communication devices that may communicate through a network), audio I/O devices 1242, and/or a data storage device 1244. Data storage device 1244 may store code 1246, which may be executed by processors 1202a and/or 1202b. In alternative embodiments, any portions of the bus architectures could be implemented with one or more PtP links.

The computer system depicted in FIG. 12 is a schematic illustration of an embodiment of a computing system that may be utilized to implement various embodiments discussed herein. It will be appreciated that various components of the system depicted in FIG. 12 may be combined in a system-on-a-chip (SoC) architecture or in any other suitable configuration. For example, embodiments disclosed herein can be incorporated into systems including mobile devices such as smart cellular telephones, tablet computers, personal digital assistants, portable gaming devices, etc. It will be appreciated that these mobile devices may be provided with SoC architectures in at least some embodiments.

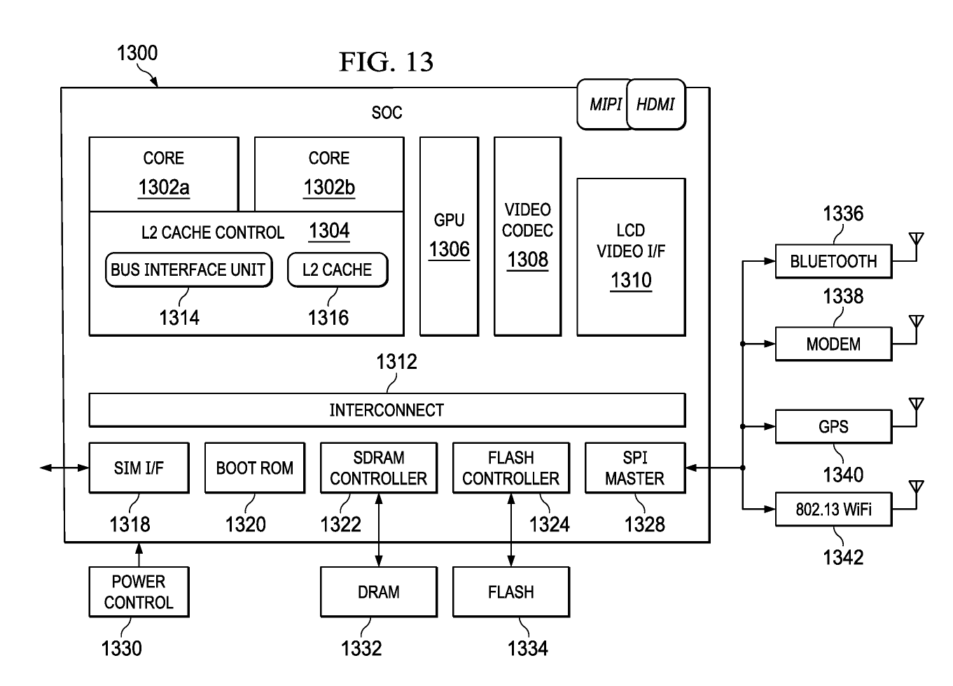

Turning to FIG. 13, FIG. 13 is a simplified block diagram associated with an example ecosystem SOC 1300 of the present disclosure. At least one example implementation of the present disclosure can include the device pairing in a local network features discussed herein and an ARM component. For example, the example of FIG. 13 can be associated with any ARM core (e.g., A-9, A-15, etc.). Further, the architecture can be part of any type of tablet, smartphone (inclusive of Android™ phones, iPhones™), iPad™, Google Nexus™, Microsoft Surface™, personal computer, server, video processing components, laptop computer (inclusive of any type of notebook), Ultrabook™ system, any type of touch-enabled input device, etc.

In this example of FIG. 13, ecosystem SOC 1300 may include multiple cores 1302a and 1302b, an L2 cache control 1304, a graphics processing unit (GPU) 1306, a video codec 1308, a liquid crystal display (LCD) I/F 1310 and an interconnect 1312. L2 cache control 1304 can include a bus interface unit 1314, a L2 cache 1316. Liquid crystal display (LCD) I/F 1310 may be associated with mobile industry processor interface (MIPI)/high-definition multimedia interface (HDMI) links that couple to an LCD.

Ecosystem SOC 1300 may also include a subscriber identity module (SIM) I/F 1318, a boot read-only memory (ROM) 1320, a synchronous dynamic random-access memory (SDRAM) controller 1322, a flash controller 1324, a serial peripheral interface (SPI) master 1328, a suitable power control 1330, a dynamic RAM (DRAM) 1332, and flash 1334. In addition, one or more embodiments include one or more communication capabilities, interfaces, and features such as instances of Bluetooth™ 1336, a 3G modem 0138, a global positioning system (GPS) 1340, and an 802.11 Wi-Fi 1342.

In operation, the example of FIG. 13 can offer processing capabilities, along with relatively low power consumption to enable computing of various types (e.g., mobile computing, high-end digital home, servers, wireless infrastructure, etc.). In addition, such an architecture can enable any number of software applications (e.g., Android™, Adobe° Flash° Player, Java Platform Standard Edition (Java SE), JavaFX, Linux, Microsoft Windows Embedded, Symbian and Ubuntu, etc.). In at least one example embodiment, the core processor may implement an out-of-order superscalar pipeline with a coupled low-latency level-2 cache.

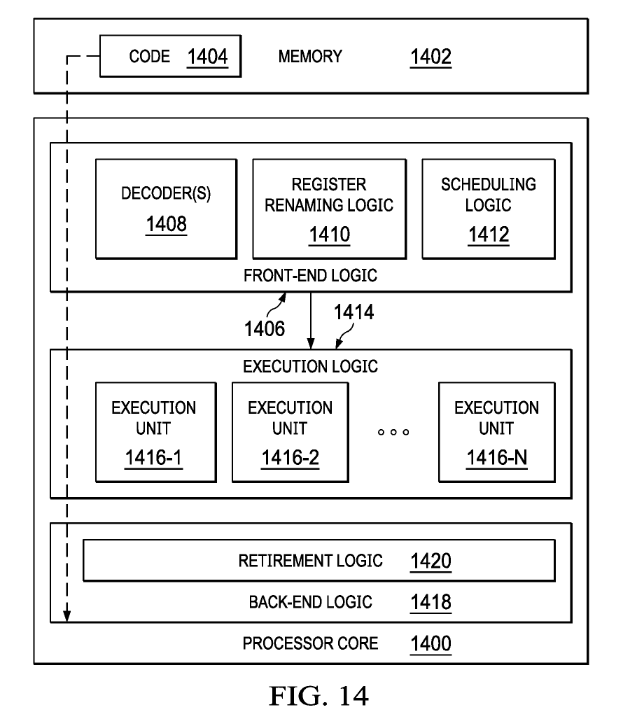

Turning to FIG. 14, FIG. 14 illustrates a processor core 1400 according to an embodiment. Processor core 1400 may be the core for any type of processor, such as a micro-processor, an embedded processor, a digital signal processor (DSP), a network processor, or other device to execute code. Although only one processor core 1400 is illustrated in FIG. 14, a processor may alternatively include more than one of the processor core 1400 illustrated in FIG. 14. For example, processor core 1400 represents one example embodiment of processors cores 1404a-1404d shown and described with reference to processors 1402a and 1402b of FIG. 14. Processor core 1400 may be a single-threaded core or, for at least one embodiment, processor core 1400 may be multithreaded in that it may include more than one hardware thread context (or “logical processor”) per core.

FIG. 14 also illustrates a memory 1402 coupled to processor core 1400 in accordance with an embodiment. Memory 1402 may be any of a wide variety of memories (including various layers of memory hierarchy) as are known or otherwise available to those of skill in the art. Memory 1402 may include code 1404, which may be one or more instructions, to be executed by processor core 1400. Processor core 1400 can follow a program sequence of instructions indicated by code 1404. Each instruction enters a front-end logic 1406 and is processed by one or more decoders 1408. The decoder may generate, as its output, a micro operation such as a fixed width micro operation in a predefined format, or may generate other instructions, microinstructions, or control signals that reflect the original code instruction. Front-end logic 1406 also includes register renaming logic 1410 and scheduling logic 1412, which generally allocate resources and queue the operation corresponding to the instruction for execution.

Processor core 1400 can also include execution logic 1414 having a set of execution units 1416-1 through 1416-N. Some embodiments may include a number of execution units dedicated to specific functions or sets of functions. Other embodiments may include only one execution unit or one execution unit that can perform a particular function. Execution logic 1414 performs the operations specified by code instructions.

After completion of execution of the operations specified by the code instructions, back-end logic 1418 can retire the instructions of code 1404. In one embodiment, processor core 1400 allows out of order execution but requires in order retirement of instructions. Retirement logic 1420 may take a variety of known forms (e.g., re-order buffers or the like). In this manner, processor core 1400 is transformed during execution of code 1404, at least in terms of the output generated by the decoder, hardware registers and tables utilized by register renaming logic 1410, and any registers (not shown) modified by execution logic 1414.

Although not illustrated in FIG. 14, a processor may include other elements on a chip with processor core 1400, at least some of which were shown and described herein with reference to FIG. 14. For example, as shown in FIG. 14, a processor may include memory control logic along with processor core 1400. The processor may include I/O control logic and/or may include I/O control logic integrated with memory control logic.

Note that with the examples provided herein, interaction may be described in terms of two, three, or more elements. However, this has been done for purposes of clarity and example only. In certain cases, it may be easier to describe one or more of the functionalities of a given set of flows by only referencing a limited number of elements. It should be appreciated that eye movement detection system 102 and its teachings are readily scalable and can accommodate a large number of components, as well as more complicated/sophisticated arrangements and configurations. Accordingly, the examples provided should not limit the scope or inhibit the broad teachings of eye movement detection system 102 as potentially applied to a myriad of other architectures.

It is also important to note that the operations in the preceding flow diagrams (i.e., FIGS. 9-11) illustrate only some of the possible correlating scenarios and patterns that may be executed by, or within, eye movement detection system 102. Some of these operations may be deleted or removed where appropriate, or these operations may be modified or changed considerably without departing from the scope of the present disclosure. In addition, a number of these operations have been described as being executed concurrently with, or in parallel to, one or more additional operations. However, the timing of these operations may be altered considerably. The preceding operational flows have been offered for purposes of example and discussion. Substantial flexibility is provided by eye movement detection system 102 in that any suitable arrangements, chronologies, configurations, and timing mechanisms may be provided without departing from the teachings of the present disclosure.

Although the present disclosure has been described in detail with reference to particular arrangements and configurations, these example configurations and arrangements may be changed significantly without departing from the scope of the present disclosure. Moreover, certain components may be combined, separated, eliminated, or added based on particular needs and implementations. Additionally, although eye movement detection system 102 has been illustrated with reference to particular elements and operations that facilitate the communication process, these elements and operations may be replaced by any suitable architecture, protocols, and/or processes that achieve the intended functionality of eye movement detection system 102.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph six (6) of 35 U.S.C. section 112 as it exists on the date of the filing hereof unless the words “means for” or “step for” are specifically used in the particular claims; and (b) does not intend, by any statement in the specification, to limit this disclosure in any way that is not otherwise reflected in the appended claims.

### OTHER NOTES AND EXAMPLES

Example M1 is a method including determining that a pressure in an ear of a user has changed, determining that the pressure change indicates that an eye of the user is going to move, predicting a direction of change of the eye of the user, and tracking at least one eye of the user based, at least partially, on the determined pressure change.

In Example M2, the subject matter of Example M1 can optionally include waking an eye tracking system from a low power state based on the determined pressure change.

In Example M3, the subject matter of any one of the Examples M1-M2 can optionally include where the eye tracking system includes a camera to track the eye of the user.

In Example M4, the subject matter of any one of the Examples M1-M3 can optionally include predicting a location of the eye of the user based on the pressure change.

In Example M5, the subject matter of any one of the Examples M1-M4 can optionally include where the pressure change in the ear is at least partially determined by using a microphone that detects sound caused by the pressure change in the ear.

In Example M6, the subject matter of any one of the Examples M1-M5 can optionally include where the microphone is in an ear canal of the user.

In Example M7, the subject matter of any one of the Examples M1-M6 can optionally include where the microphone is outside an ear canal of the user.

Example C1 is at least one machine readable medium having one or more instructions that when executed by at least one processor cause the at least one processor to determine that a pressure in an ear of a user has changed, determine that the pressure change indicates that an eye of the user is going to move, cause a camera to exit a low power state and enter an active power state, and track at least one eye of the user based, at least partially, on the determined pressure change.

In Example C2, the subject matter of Example C1 can optionally include one or more instructions that when executed by the least one processor, causes the least one processor to wake an eye tracking system from a low power state based on the determined pressure change.

In Example C3, the subject matter of any one of Examples C1-C2 can optionally include one or more instructions that when executed by the at least one processor, further cause the processor to predict a direction of change of the eye of the user.

In Example C4, the subject matter of any one of Examples C1-C3 can optionally include one or more instructions that when executed by the least one processor, causes the least one processor to predict a location of the eye of the user based on the pressure change.

In Example C5, the subject matter of any one of Examples C1-C4 can optionally include where the pressure change in the ear is at least partially determined by using a microphone that detects sound caused by the pressure change in the ear.

In Example C6, the subject matter of any one of Examples C1-C5 can optionally include where the microphone is in an ear canal of the user.

In Example C7, the subject matter of any one of Example C1-C6 can optionally include where the microphone is outside an ear canal of the user.

Example S1 is an eye movement detection including a an ear pressure based eye movement detection engine and an eye tracking engine. The ear pressure based eye movement detection engine is configured to determine that a pressure in an ear of a user has changed, determine that the pressure change indicates that an eye of the user is going to move, and predict a direction of change of the eye of the use. The eye tracking engine is configured to track the eye of the user based, at least partially, on the determined pressure change.

In Example S2, the subject matter of Example S1 can optionally include where the ear pressure based eye movement detection engine is further configured to wake the eye tracking engine from a low power state based on the determined pressure change.

In Example S3, the subject matter of any of the Examples S1-S2 can optionally include where the ear pressure based eye movement detection engine is further configured to predict a location of the eye of the user based on the pressure change.

In Example S4, the subject matter of any of the Examples S1-S3 can optionally include where the pressure change in the ear is at least partially determined by using a microphone that detects sound caused by the pressure change in the ear.

In Example S5, the subject matter of any of the Examples S1-S4 can optionally include where the microphone is in an ear canal of the user.

In Example S6, the subject matter of any of the Examples S1-S5 can optionally include where the microphone is outside an ear canal of the user.

In Example A1, an electronic device can include means for determining that a pressure in an ear of a user has changed, means for determining that the pressure change indicates that an eye of the user is going to move, means for predicting a direction of change of the eye of the user, and means for tracking the eye of the user based, at least partially, on the determined pressure change.

In Example, A2, the subject matter of Example A1 can optionally include means for waking an eye tracking system from a low power state based on the determined pressure change.

In Example A3, the subject matter of any one of Examples A1-A2 can optionally include where the eye tracking system includes a camera to track the eye of the user.

In Example A4, the subject matter of any one of Examples A1-A3 can optionally include means for predicting a location of the eye of the user based on the pressure change.

In Example A5, the subject matter of any one of Examples A1-A4 can optionally include where the pressure change in the ear is at least partially determined by using a microphone that detects sound caused by the pressure change in the ear.

In Example A6, the subject matter of any one of Examples A1-A5 can optionally include where the microphone is in an ear canal of the user.

In Example A7, the subject matter of any one of Examples A1-A6 can optionally include where the microphone is outside an ear canal of the user.

Example X1 is a machine-readable storage medium including machine-readable instructions to implement a method or realize an apparatus as in any one of the Examples A1-A7, or M1-M7. Example Y1 is an apparatus comprising means for performing of any of the Example methods M1-M7. In Example Y2, the subject matter of Example Y1 can optionally include the means for performing the method comprising a processor and a memory. In Example Y3, the subject matter of Example Y2 can optionally include the memory comprising machine-readable instructions.

What is claimed is:

1. A method comprising:
determining that a pressure in an ear of a user has changed;
determining that the pressure change indicates that an eye of the user is going to move;
predicting a direction of change of the eye of the user; and
tracking at least one eye of the user based, at least partially, on the determined pressure change.

2. The method of claim 1, further comprising:
waking an eye tracking system from a low power state based on the determined pressure change.

3. The method of claim 2, wherein the eye tracking system includes a camera to track the eye of the user.

4. The method of claim 1, further comprising:
predicting a location of the eye of the user based on the pressure change.

5. The method of claim 1, wherein the pressure change in the ear is at least partially determined by using a microphone that detects sound caused by the pressure change in the ear.

6. The method of claim 5, wherein the microphone is in an ear canal of the user.

7. The method of claim 5, wherein the microphone is outside an ear canal of the user.

8. At least one computer-readable medium comprising one or more instructions that when executed by at least one processor, cause the at least one processor to:
determine that a pressure in an ear of a user has changed;
determine that the pressure change indicates that an eye of the user is going to move;
cause a camera to exit a low power state and enter an active power state; and
track at least one eye of the user based, at least partially, on the determined pressure change.

9. The at least one computer-readable medium of claim 8, further comprising one or more instructions that when executed by the at least one processor, further cause the processor to:
wake an eye tracking system from a low power state based on the determined pressure change.

10. The at least one computer-readable medium of claim 9, further comprising one or more instructions that when executed by the at least one processor, further cause the processor to predict a direction of change of the eye of the user.

11. The at least one computer-readable medium of claim 8, further comprising one or more instructions that when executed by the at least one processor, further cause the processor to:
predict a location of the eye of the user based on the pressure change.

12. The at least one computer-readable medium of claim 8, wherein the pressure change in the ear is at least partially determined by using a microphone that detects sound caused by the pressure change in the ear.

13. The at least one computer-readable medium of claim 12, wherein the microphone is in an ear canal of the user.

14. The at least one computer-readable medium of claim 12 wherein the microphone is outside an ear canal of the user.

15. An eye movement detection system comprising:
an ear pressure based eye movement detection engine, wherein the ear pressure based eye movement detection engine is configured to:
determine that a pressure in an ear of a user has changed;
determine that the pressure change indicates that an eye of the user is going to move; and
predict a direction of change of the eye of the user; and
an eye tracking engine, wherein the eye tracking engine is configured to:
track the eye of the user based, at least partially, on the determined pressure change.

16. The eye movement detection system of claim 15, wherein the ear pressure based eye movement detection engine is further configured to:
wake the eye tracking engine from a low power state based on the determined pressure change.

17. The eye movement detection system of claim 15, wherein the ear pressure based eye movement detection engine is further configured to:
predict a location of the eye of the user based on the pressure change.

18. The eye movement detection system of claim 15, wherein the pressure change in the ear is at least partially determined by using a microphone that detects sound caused by the pressure change in the ear.

19. The eye movement detection system of claim 18, wherein the microphone is in an ear canal of the user.

20. The eye movement detection system of claim 18, wherein the microphone is outside an ear canal of the user.
